SEQUENCE LISTING (14 sequences)

SEQ ID NO: 1 - DNA, 2709 bp, Populus balsamifera subsp. trichocarpa
  CDS (37)..(2706)
cagccaaatcaaacccaccacccccgaaatcggaaaatgtcctccgcccttggctaccaccatcaccaccacctacaacaagaacaacaacaacaacaacagaaacccttctccgactccgattcctactccgactccgactccgacaccaccaacagccaaaaccaacactccgcagatctcacaaactcaatcttcaaatcctacttcgaacacgccaaccaccaatccttacaaccaacgcaacacgacctaaccaaaatcaaatccttcctaacctcctcctcctctggcgctctatcatgtctcatctgtctcgaacgcatcaaaacctccgatccgacctggtcttgcacttctctctgctacgccgttttccaccttatctgtatccaatcctgggctcgccaagcctccgatctctccgctcttcgagcctccacgcgcctccctatctcctctgataaagccgctgaatcctccacctggaattgccctaaatgccgctctgattattctagatctaaaatccccagaaattacctctgtttctgtggcaaagtcgaaaatccaccaaatgatccttggattttatcccattcttgcggtgaaatctgtaaccgtcagttgaagaataactgcggtcattgctgtttgttactttgccatcccggtccctgcccatcttgcccgaaacttgtcaaagcgacatgtttttgcggcaaaacgacggatgttaagcgttgcggttacaaattattttcttgtaacaatatttgtaaaaaatcattagattgcgggattcatagctgtaaacaaatttgtcacgatggaccctgcccgccgtgtaatgcccgcggggtttataaatgttcatgtggaaggaaagttgaggagagggagtgttgtgagagagaatttagatgtgagaatccctgtgagaaattgttagcctgcggaaaacatgtgtgtgagagggggtgccattttggggagtgtggagattgtcctcttcaagggaagagagcatgtccatgcgggaagagactctatgaaggaatggcttgtgatattgttgtgccgctttgtggtggtacctgtgataaaatgttgagttgtggtttccataggtgccacgagagatgtcatcgagggccttgcattgagacttgtcgaattgttgtcaccaagttgtgtaggtgtggggggatgaagaaagaggttccttgctatcaagatttggcatgtgagaggaagtgtcaaagaatgagagattgtggacgacatgcttgtaaacgtcgctgctgtgatggggactgtccgccatgtggagagatttgcggcaagaggcttcggtgtaagaatcacaagtgtcctgccccgtgtcatcgaggtgcctgttctccttgtcctgtaatgtttacaatttcatgtgcttgtggagagactcattttgaggttccctgtggcactgagaaagaccaaaagcctccaaaatgtcgtaaatcatgtggaatatctcctttatgccgacatggatcagattctaagccacacaaatgccattatggggcatgccctccttgtcgattgctttgtgatgaagaatacccatgcagccataagtgcaaattaaggtgccacggccccagaccacctcctaatccagatttcacattgaggccaaagaaaaagaaaccaaatcatcagagtgaaagtacaccaggaaccccatgccctccttgccctgaacttgtttggaggccatgtcttggccagcacattggagcagagagaatgatggtctgctcaaacagaacccaattttcctgtgaaaatctgtgtgggagccctctttcttgtggcaatcattattgtacaaaaacttgccatgcactgaagagccagtcttcaacttcattggttcaacataaaagaagcgagtcttgtgaagagtgtcatcttccttgtgaaaaggaacggaaacctgcttgtcgacattcttgccctctaccatgtcatcctggggactgccctccttgcaaagttcttgtgaaacgatcttgttattgtggttctatggtccacgtttttgagtgcatatattacaataacttgtcagaaaaggagcaaatggctgctcgttcatgtggtggatcttgccataggaaattgcctaattgtacacatttatgtcccaagacatgtcatccaggtcaatgcccatcacctgacaagtgcgccaaaaaggtcactgttcgttgtcaatgccaaaccttgaaaaaggagatgccatgtcaagaggttcaagcagcctatcacaaagcaggttccgatcccaaagatatatctaaaagtcattttggacttggacttcttccttgcaattctggttgcaagagtaaagcacaggttgtggatcaggaattgcatttgcgtaaatccaaagacctggaggagaaggttccagccactgaaattcatccaccgaagcgccgaaaacggcgtgaacatttacaagaaacaaagaagacctcacaactgcagaaaattgctgccaccatgaagtggcttcttgtaatagtcacccttatggtgactatggtggcagctgcatatttcggttacaaaggtctcatttggctctccgattggatgaatgaagttgaagagcaacaaagacacagaagaagacatccacgaatctaa

SEQ ID NO: 2 - protein, 890 aa, Populus balsamifera subsp. trichocarpa
MSSALGYHHHHHLQQEQQQQQQKPFSDSDSYSDSDSDTTNSQNQHSADLTNSIFKSYFEHANHQSLQPTQHDLTKIKSFLTSSSSGALSCLICLERIKTSDPTWSCTSLCYAVFHLICIQSWARQASDLSALRASTRLPISSDKAAESSTWNCPKCRSDYSRSKIPRNYLCFCGKVENPPNDPWILSHSCGEICNRQLKNNCGHCCLLLCHPGPCPSCPKLVKATCFCGKTTDVKRCGYKLFSCNNICKKSLDCGIHSCKQICHDGPCPPCNARGVYKCSCGRKVEERECCEREFRCENPCEKLLACGKHVCERGCHFGECGDCPLQGKRACPCGKRLYEGMACDIVVPLCGGTCDKMLSCGFHRCHERCHRGPCIETCRIVVTKLCRCGGMKKEVPCYQDLACERKCQRMRDCGRHACKRRCCDGDCPPCGEICGKRLRCKNHKCPAPCHRGACSPCPVMFTISCACGETHFEVPCGTEKDQKPPKCRKSCGISPLCRHGSDSKPHKCHYGACPPCRLLCDEEYPCSHKCKLRCHGPRPPPNPDFTLRPKKKKPNHQSESTPGTPCPPCPELVWRPCLGQHIGAERMMVCSNRTQFSCENLCGSPLSCGNHYCTKTCHALKSQSSTSLVQHKRSESCEECHLPCEKERKPACRHSCPLPCHPGDCPPCKVLVKRSCYCGSMVHVFECIYYNNLSEKEQMAARSCGGSCHRKLPNCTHLCPKTCHPGQCPSPDKCAKKVTVRCQCQTLKKEMPCQEVQAAYHKAGSDPKDISKSHFGLGLLPCNSGCKSKAQVVDQELHLRKSKDLEEKVPATEIHPPKRRKRREHLQETKKTSQLQKIAATMKWLLVIVTLMVTMVAAAYFGYKGLIWLSDWMNEVEEQQRHRRRHPRI

SEQ ID NO: 3 - DNA, 1692 bp, Populus balsamifera subsp. trichocarpa
  CDS (1)..(1689)
atgctcacgtggataacacgtgacttcattaccccgccccttgttcgttcttcaatgccagccgaaccaaaccccactccagttccactgctccagagtccagctccccaaattctgaaatgtcctctgcccacagccaccatcaacaccgacagctgttctccgaatccgtcaccaccactctgcagatctcacaaactcaattttcaaatcctatcatgtctcatttctctcgaagcatcaaaccatccgatccaacctggtcttccacttctctatgctgcgccgttttcgaccttatctggtatcgaatcctggacccgccaatcctccgatctctccgctcttcgagcctccacgcgcctccctgcctcccccgataaagccgccgaatcatccacctgcatctgccctaaatgccgctctgaatattccatatctcaaattccaagaagttacctctgtttctgtggcaaagtggaaaatccaccacatgataatccccggattttaactcactcttgctgcgaaatctgttaccgaccgttgaagaataactgtggttatttctgtttgttgctatgccatcccgttccttcccatcttgcccgaaacttgtcaaaacgacatgtttttgtggcaaaacgatggatgttaagctgcagttacaaattgttttcctgtaacaatgtctgtaataaatcattggattgtgggattcataattgtaagcaaatctgccatgatggaacttgcctactgtgtaatggccgcggggtttataaatgtttatgtggaagggaagtcgaggagaggggagtgttagaggttgccgttttggggatgagagtttatgaaggattggcttgtgatgttgttgtgccgctttgtggtggtacttgtgataaaatgccgagttggggtttgcataggttccatgagagatgtcatggaaggcattgcattgagacttgtcgaattgttgtcatcaagttgtgtcagtgtgggaagatgaagatagagatttgtggcaagaggcttcagtgtgagaatcataaaggtgcctgtgctccttgtactgtcatgtttacaatttcatgtgcatgtggagatactcactttgaggttcaaaatcagaaagtcaatgaatggagtccaccaaatctatctctcatgattttaagactctttagctctaatagggaactgcatgattgtccacatttatgtcacgagacatgttgtccaggtcaatgcccatcacctgacaagtgcaccaaagtatactgcaaagcaggctctgatcccaaagatatatctgaaaaccattttggacctgaacttcttccttggaattatgcttgcaagagtaaagcacaggtcgtggatcaggaattgcatttgtgtaaatccaaagatttggaggaaaaggttccagctactaaaaatcatgtacctaagcaccaaaatcggcgtgaacgactgcaagagcaaagaagacctcacaaatgcgggtatgttactcttctgaagcaaagaccaacagaagatgctgccactatgaggtggctttttctaatggtcacccttgtggtgactctggtggcagcagcatattttggttacaaaggtctcatgtggctctctgattggatggatgaagttgaagagcaacgagacagagaaccatga

SEQ ID NO: 4 - protein, 563 aa, Populus balsamifera subsp. trichocarpa
MLTWITRDFITPPLVRSSMPAEPNPTPVPLLQSPAPQILKCPLPTATINTDSCSPNPSPPLCRSHKLNFQILSCLISLEASNHPIQPGLPLLYAAPFSTLSGIESWTRQSSDLSALRASTRLPASPDKAAESSTCICPKCRSEYSISQIPRSYLCFCGKVENPPHDNPRILTHSCCEICYRPLKNNCGYFCLLLCHPVPSHLARNLSKRHVFVAKRWMLSCSYKLFSCNNVCNKSLDCGIHNCKQICHDGTCLLCNGRGVYKCLCGREVEERGVLEVAVLGMRVYEGLACDVVVPLCGGTCDKMPSWGLHRFHERCHGRHCIETCRIVVIKLCQCGKMKIEICGKRLQCENHKGACAPCTVMFTISCACGDTHFEVQNQKVNEWSPPNLSLMILRLFSSNRELHDCPHLCHETCCPGQCPSPDKCTKVYCKAGSDPKDISENHFGPELLPWNYACKSKAQVVDQELHLCKSKDLEEKVPATKNHVPKHQNRRERLQEQRRPHKCGYVTLLKQRPTEDAATMRWLFLMVTLVVTLVAAAYFGYKGLMWLSDWMDEVEEQRDREP

SEQ ID NO: 5 - protein, 161 aa, Populus balsamifera subsp. trichocarpa
KVENPPNDPWILSHSCGEICNRQLKNNCGHCCLLLCHPGPCPSCPKLVKATCFCGKTTDVKRCGYKLFSCNNICKKSLDCGIHSCKQICHDGPCPPCNARGVYKCSCGRKVEERECCEREFRCENPCEKLLACGKHVCERGCHFGECGDCPLQGKRACPCG

SEQ ID NO: 6 - DNA, 3798 bp, Populus balsamifera subsp. trichocarpa
  CDS (1)..(3336)
atgagcttccagcccagaaatgacgggggagataataataatggtagcaggtccagattcccgacccgacaaacgtgggttcccagaggatctaatccttctcttccactgaatggggatgtaaacccaaacccaaacccaaacccaaacccaaacccaccttcgagtttcagctctagaaacaatggaaatggtgggcattctagccatgggactggagtagctgattataggtataagggtggtgtgaatgcacctagaggtggtcaaatgggtcgggggaaggagaggggggtggagactcgggaagtgaaggacccgaatttgccgcaacttgcgcaagaaattcaggagaagcttttgaagagtacggttgagtgtatgatttgttatgatatggttcggaggtctgcgcctgtttggtcttgttcgagttgtttctcgatttttcacttgaattgtatcaagaaatgggctcgtgcgccgacgtctgttgatttgattgcggagaagaatcaaggttttaattggcggtgtccgggatgtcaatctgtgcagcttacgtcgttgaaggatattcggtatgtttgcttttgtgggaagaggacggatccgccttcggatttgtatttgaccccgcattcatgtggggaaccgtgtggaaaacagcttgagaaggaggttcctggtgctgatgggagtagggagggtctttgtccgcacaattgtgtcttgcagtgtcaccctgggccatgtcctccttgtaaggcctttgctccaccaagtctgtgtccttgtgggaagaaaagaatcacaacaagatgtgctgatcggaagtccgttcttacctgtgggcaacgatgtgataagcttctggaatgttggcgtcatcgatgtgagcagatttgccatgttggtccctgcaatccttgtcaggtattgattaatgcttcatgtttctgcaagaagaacacggaggttgttctatgtggggatatggcagtgaagggagaagtgaaagctgaagatggtgttttttcttgcaactcaacatgtggaaaggttctgggttgtggaaatcatatttgtggtgaaacttgtcatccaggtgattgcggggattgtgagtttatgcctggcagggtcaagtcatgctattgtggaaaaacaagtttgcaggaggaaagaaatagttgtttggatccaattccaacgtgtgcacaaatatgtggcaaatcccttccttgcgggatgcaccaatgtaaagaggtgtgccattcaggggattgtgctccttgtttggtttctgtcacccaaaaatgccgctgcggatcaacctcccgaactgtggagtgttacaaaactacttcagagaatgaaaaattcctgtgtgacaagccttgtggtcgaaagaagaactgtggaaggcataggtgcagcgagcgatgctgccctctttccaactctaacaatcagttctctggggattgggatccacatttttgtcaaatggcttgtgggaagaagttaaggtgtggacaacactcttgtgaatcactgtgtcacagtggccattgtcctccttgtcttgaaacaattttcactgatttgacatgtgcatgtgggagaacttccatccctcctccattgccgtgcggcacgcctcccccttcatgtcagctcccatgctcagttcctcagccatgtggccatccagcttctcatagctgccacttcggagactgcccaccttgttcggtgcctgtagctaaagagtgtgttggtgggcatgtgattctggggaacataccctgcgggtcaagggatatcagatgcaacaaactctgtgggaagaccaggcaatgtggtttgcatgcatgtggaagaacttgtcattcaccgccttgtgatacctctcctggaactgaaactggctcaagagcttcttgtgggcagacatgcggtgcccctaggagagattgccggcatacttgcactgcactttgtcatccttatgctccttgtcctgatgtcagatgtgagttccctgtcacaattacttgctcttgcggcaggatgacagcatctgttccttgtgatgctggagggagcaatggtggttataatgatactattttagaagcatccattctccacaagttgccagcgcctcttcaaccagtggaatcatctggaaagaagattcctcttggccagagaaagtttatgtgtgatgatgaatgtgccaagtttgagcggaaaagggttcttgcagatgcttttgatatcaaccctccaaatttggaagctcttcattttggtgagaattcttccgtgacagagttgattggtgatctgtacaggcgcgatccaaaatgggtgcttgctgtggaggagaggtgcaaatatttggttcttagcaagagcagaggaaccacaagtggtcttaagattcatgttttctgcccaatgctgaaggataaaagagatgcagttaggctaattgctgagaggtggaaggttgcaatttattctgctggttgggagccaaagcgttttattgtaatccatgctacaccaaaatccaaaaccccatctcgtgtgattggcattaagggtacgacaactttaagtgcctcccatccaccagtttttgatgctctcgtagacatggatcccaggcttgttgtttcttttctagatttgccgagggaggcggatataagctcactggtccttaggtttggtggggaatgcgaactagtttggttgaatgataagaatgccctggctgtattcaatgatcctgctcgagcagcaaccgctatgaggaggttggatcatggatcagtatattatggagctgcggtggttcctcaaaatagcggtgcatccatgggatcaccagctaccaatgcctggggaacggcaggcactgcaaaggaagggacgatcacagctctgaagggaacttcgtggaagaaagctgtagtgcaggagtctggttggagggaagattcatggggtgatgaagagtggtctggtggtggttctgcagatgttcaggcttctgcatggaaagggaaagaacatccaatctccacttcaattaaccgatggagtgtactggactccgataaggctgacagttcatctgctgcctctgtcaggatcgaggatcctgcaaaacgagttgcagaaattctttccagttcaggtttggaatcaaatgtaagcacttcgaatatatcagtgcaaactgcgatgcaacctggaggggtttcaagtgaagaagatttatctgaggtggtggatgactgggagaaggcctatgattgagaacatgcgtgtggagtcatctgattttttgaacatgcttccagggtatcttttggtctatacctatttcactttaaacattttggcgtttgcaggagtattttacccgttatttttatgaaaagctatatcatccacccctattgatgcttgatggtgggtgtttcttcttcttcctttcttcttttttttttccggctctctacttgaaatcccattaatttcggcggagactggagagagcatacatctttgctgtatgggagaatacttgttactgtagaactttgtcaaagaatctaggaaaaaaacatcgcggcctcgtcagcgggttttgttgatatattttcgagaaaggcattctatgtgtacagagaaaaactgagatttcgctacttttgattccaatgaaatatctgctctattttggcatgacaggtgtttcttcgttttcttttc

SEQ ID NO: 7 - protein, 1112 aa, Populus balsamifera subsp. trichocarpa
MSFQPRNDGGDNNNGSRSRFPTRQTWVPRGSNPSLPLNGDVNPNPNPNPNPNPPSSFSSRNNGNGGHSSHGTGVADYRYKGGVNAPRGGQMGRGKERGVETREVKDPNLPQLAQEIQEKLLKSTVECMICYDMVRRSAPVWSCSSCFSIFHLNCIKKWARAPTSVDLIAEKNQGFNWRCPGCQSVQLTSLKDIRYVCFCGKRTDPPSDLYLTPHSCGEPCGKQLEKEVPGADGSREGLCPHNCVLQCHPGPCPPCKAFAPPSLCPCGKKRITTRCADRKSVLTCGQRCDKLLECWRHRCEQICHVGPCNPCQVLINASCFCKKNTEVVLCGDMAVKGEVKAEDGVFSCNSTCGKVLGCGNHICGETCHPGDCGDCEFMPGRVKSCYCGKTSLQEERNSCLDPIPTCAQICGKSLPCGMHQCKEVCHSGDCAPCLVSVTQKCRCGSTSRTVECYKTTSENEKFLCDKPCGRKKNCGRHRCSERCCPLSNSNNQFSGDWDPHFCQMACGKKLRCGQHSCESLCHSGHCPPCLETIFTDLTCACGRTSIPPPLPCGTPPPSCQLPCSVPQPCGHPASHSCHFGDCPPCSVPVAKECVGGHVILGNIPCGSRDIRCNKLCGKTRQCGLHACGRTCHSPPCDTSPGTETGSRASCGQTCGAPRRDCRHTCTALCHPYAPCPDVRCEFPVTITCSCGRMTASVPCDAGGSNGGYNDTILEASILHKLPAPLQPVESSGKKIPLGQRKFMCDDECAKFERKRVLADAFDINPPNLEALHFGENSSVTELIGDLYRRDPKWVLAVEERCKYLVLSKSRGTTSGLKIHVFCPMLKDKRDAVRLIAERWKVAIYSAGWEPKRFIVIHATPKSKTPSRVIGIKGTTTLSASHPPVFDALVDMDPRLVVSFLDLPREADISSLVLRFGGECELVWLNDKNALAVFNDPARAATAMRRLDHGSVYYGAAVVPQNSGASMGSPATNAWGTAGTAKEGTITALKGTSWKKAVVQESGWREDSWGDEEWSGGGSADVQASAWKGKEHPISTSINRWSVLDSDKADSSSAASVRIEDPAKRVAEILSSSGLESNVSTSNISVQTAMQPGGVSSEEDLSEVVDDWEKAYD

SEQ ID NO: 8 - DNA, 3324 bp, Populus balsamifera subsp. trichocarpa
  CDS (1)..(3321)
atgagctttcaaccccgaaatgatcgaagagacaacaacaacaacaggtcgtctagatttcccacccagaagtgggttccgagaggagctaattctagtcctgcggttgatgcaaacaccaaaccgccttcgagttccaactctagatgcaatggaaatggtgggggtggtgctgctcatgggtggtcaggaacagcacatcataggtataataaaggtggtatggctgtgaatgcacctagaggtcttgtgggtcggcctaggaaggggatagagaggagtgagaagactcgggaattgaatgacccgaatttgccgcagcttgcacaagatattcaggagaagcttgtgaagagtacggttgagtgtatgatttgttatgatatggttcggaggtctgtgcctatttggtcttgttcgagttgtttctcgatttttcatttgaattgtatcaagaaatgggctcgtgccccgacttctgttgatttgattgcggagaagaatcaaggttttaattggcggtgtccgggatgtcaatctgtgcagcttacgacgttgaatgatattcggtatgtttgcttttgtgggaagaggagggatccaccttcggatttgtatttgactcctcattcgtgtggagaaccttgtggaaagccacttgagaaggaggctcctggtgctgatgggagtaaggaggatctttgccctcataattgtgtcttgcagtgtcaccctgggccatgtcctccttgcaaggcctttgctccaccacgcctgtgtccttgtgggaagaaaataatcacaacaagatgtgctgatcggatgtctgttgttacctgtggacacccatgtgataagcttcttgaatgttggcgtcatcgatgtgagcggatttgccatgttggtccctgcgattcttgtcaggtgttggtcaatgcttcatgtttctgcaagaaaaagacggaggttgttctgtgtggtgacatggcagtgaagggagaagtgaaagctgaagatggtgttttttcttgcaattctacatgtggaaagatgctgggttgtggaaaccatatgtgtgatgaaacttgtcatccaggtctttgtggcgattgtgagttaatgcctgccagggtcaggtcttgctattgtggaaaaacaagtttgcaggaggaaagaaagagttgtttggatccaattccaacatgtacacaaatatgcggcaaatcacttccttgcgggatgcaccaatgtaaaggggtgtgccattctggggattgcgcaccttgtttggtttctgtaacccaaaaatgccgttgtggatcaacctctcaaattgtggagtgttacaaaattacttcagagaatgaaaaattcttgtgtgagaagccttgtggtcgaaagaagaactgtggaaggcacaggtgtagcgagcgatgctgccctctttccaacactaacaatcagttctctggtgattgggatccgcacttttgtcaaatggcctgtgggaagaagttaaggtgtggacagcattcttgtgatgatctttgtcacagtggccattgtcctccttgtcttgaaacaattttcactgatttgacatgtgcgtgtaggagaacttccatccctcctccattgccttgtggtacacctcccccttcatgtcagctcccatgctcagtccctcagccttgtggccatccagcttctcatagctgtcacttcggagactgcccatcttgtttggtgcctgtagctaaggagtgtgttggtgggcatgtgattctcgggaacataccctgtgggtcaagggatatcagatgcaacaaactatgtggtaagacaaggcaatgtggtctgcatgcatgtggaagaacttgtcattcactgccttgtgatacctcatctggaaacgaaactggcacgagagcttcttgtgggcagacatgtggtgcccctaagagagattgcaggcatacttgcactgcactttgtcatcctcatgctccttgtcctgatgtcagatgtgagttccttgtcacaattagttgctcttgtggcaggatgacagcatctgttccttgtgatgctggggggagcaatggtgcttataatgacactgttttagaagcttccattctccacaagttgccagcatctcttcaaccagtggaatcaactggaaagaagattcctcttggccaaagaaagcttatgtgtgatgatgaatgtgccaagttggagaggaaaagggttcttgctgatgcttttgatatcacccctcctaatctggaagctcttcattttggtgagaattctgcagtgacagagttgattggtgatctgtacaggcgtgatccaaagtgggtgcttgctgtggaggaaaggtgcaaatatttggttcttggcaagagcagaggaaccactagtggtcttaagattcatgttttttgccctatgctgaaggataaaagggatgcagttagtctgattgctgagaggtggaagcttgcaatttattctgctggttgggaaccaaagcgtttttttgtagttcatgctacatccaaatccaaacctccacctcgtgtgattgggattaagggtacgacaactttaagttcccatccaccagtttttgatgttcttgtagacatggatcccaggcttgttgtatcttttctagatttgccgagggaggcagatataagttcactggtccttaggtttggtggggaatgtgaattagtttggttgaatgataagaatgcactggctgtattcaatgatcctgctcgagcagctactgctatgagaaggctggatcatggatcattatatcatggagcatctgtggttcctcaaaatactggtgcatctgtggcatcaccagctaataatgcctgggcagtggcagggactgcaatggaaggaacagttgcagctttgaagggaacttcgtggaagaaagctgtagtgcaggagactggttgtaagaaatattcatggagtggtgaagagtggtctgatggtggttctgcagatgttcaggcttctgcatggaaagggaaagaagctccaattgtggcttcgattaaccgatggagtgtcctagactctgagaaggctgacagttcatctgctgcctctgtcaagatggaagatcccgcaaaacaagttgcaggaagcctttccagttcaggtttggaatcaaatgcaagcacttccagtgcatcaaggcagcctgcaatgcaatctggaggagtttcaagagaagaagatttatctgtggtggtggatgattgggagaaggcttatgattga

SEQ ID NO: 9 - protein, 1107 aa, Populus balsamifera subsp. trichocarpa
MSFQPRNDRRDNNNNRSSRFPTQKWVPRGANSSPAVDANTKPPSSSNSRCNGNGGGGAAHGWSGTAHHRYNKGGMAVNAPRGLVGRPRKGIERSEKTRELNDPNLPQLAQDIQEKLVKSTVECMICYDMVRRSVPIWSCSSCFSIFHLNCIKKWARAPTSVDLIAEKNQGFNWRCPGCQSVQLTTLNDIRYVCFCGKRRDPPSDLYLTPHSCGEPCGKPLEKEAPGADGSKEDLCPHNCVLQCHPGPCPPCKAFAPPRLCPCGKKIITTRCADRMSVVTCGHPCDKLLECWRHRCERICHVGPCDSCQVLVNASCFCKKKTEVVLCGDMAVKGEVKAEDGVFSCNSTCGKMLGCGNHMCDETCHPGLCGDCELMPARVRSCYCGKTSLQEERKSCLDPIPTCTQICGKSLPCGMHQCKGVCHSGDCAPCLVSVTQKCRCGSTSQIVECYKITSENEKFLCEKPCGRKKNCGRHRCSERCCPLSNTNNQFSGDWDPHFCQMACGKKLRCGQHSCDDLCHSGHCPPCLETIFTDLTCACRRTSIPPPLPCGTPPPSCQLPCSVPQPCGHPASHSCHFGDCPSCLVPVAKECVGGHVILGNIPCGSRDIRCNKLCGKTRQCGLHACGRTCHSLPCDTSSGNETGTRASCGQTCGAPKRDCRHTCTALCHPHAPCPDVRCEFLVTISCSCGRMTASVPCDAGGSNGAYNDTVLEASILHKLPASLQPVESTGKKIPLGQRKLMCDDECAKLERKRVLADAFDITPPNLEALHFGENSAVTELIGDLYRRDPKWVLAVEERCKYLVLGKSRGTTSGLKIHVFCPMLKDKRDAVSLIAERWKLAIYSAGWEPKRFFVVHATSKSKPPPRVIGIKGTTTLSSHPPVFDVLVDMDPRLVVSFLDLPREADISSLVLRFGGECELVWLNDKNALAVFNDPARAATAMRRLDHGSLYHGASVVPQNTGASVASPANNAWAVAGTAMEGTVAALKGTSWKKAVVQETGCKKYSWSGEEWSDGGSADVQASAWKGKEAPIVASINRWSVLDSEKADSSSAASVKMEDPAKQVAGSLSSSGLESNASTSSASRQPAMQSGGVSREEDLSVVVDDWEKAYD

SEQ ID NO: 10 - protein, 191 aa, Populus balsamifera subsp. trichocarpa
PPNLEALHFGENSSVTELIGDLYRRDPKWVLAVEERCKYLVLSKSRGTTSGLKIHVFCPMLKDKRDAVRLIAERWKVAIYSAGWEPKRFIVIHATPKSKTPSRVIGIKGTTTLSASHPPVFDALVDMDPRLVVSFLDLPREADISSLVLRFGGECELVWLNDKNALAVFNDPARAATAMRRLDHGSVYYGA

SEQ ID NO: 11 - DNA, 30 bp, Artificial
  PCR primer
caccgcggccgcccatctcgtgtgattggc

SEQ ID NO: 12 - DNA, 23 bp, Artificial
  PCR primer
cttccacgaagttcccttcagag

SEQ ID NO: 13 - DNA, 30 bp, Artificial
  PCR primer
caccgcggccgcggacttggacttcttcct

SEQ ID NO: 14 - DNA, 24 bp, Artificial
  PCR primer
gattcgtggatgtcttcttctgtg

(19) United States
(12) Patent Application Publication
(10) Pub. No.: US 2015/0033389 A1
(43) Pub. Date: Jan. 29, 2015

(54) PLANTS HAVING IMPROVED GROWTH PROPERTIES

(71) Applicant: SweTree Technologies AB, Umeå (SE)

(72) Inventors: Maria Eriksson, Umea (SE); Naoki Takata, Takahagi (JP); Mikael Johansson, Umea (SE)

(21) Appl. No.: 14/382,937

(22) PCT Filed: Mar. 5, 2013
(86) PCT No.: PCT/SE2013/050191

§ 371 Date: Sep. 4, 2014

Related U.S. Application Data
(60) Provisional application No. 61/607,404, filed on Mar. 6, 2012.

(51) Int. Cl. C12N 15/82; A01H 1/02

(52) U.S. Cl. CPC C12N 15/8205; A01H 1/02; C12N 15/8261
USPC 800/260; 800/290; 800/298

(57) ABSTRACT

The invention relates to a method for producing a genetically modified plant with improved growth properties as compared to a corresponding non-genetically modified wild type plant, said method comprising reducing or deleting the amount or activity of an EBI1 or EBI2 polypeptide in a plant cell, a plant or a part thereof.

### TECHNICAL FIELD

The invention relates to a method for producing a genetically modified plant with improved growth properties as compared to a corresponding non-genetically modified wild type plant, said method comprising reducing or deleting the amount or activity of an EBI1 or EBI2 polypeptide in a plant cell, a plant or a part thereof.

### BACKGROUND ART

Plants use light-dark cues and an internal 24-h (circadian) clock to orient themselves in their local environment and to synchronize their metabolism accordingly. The circadian clock of the model plant Arabidopsis (Arabidopsis thaliana) is made up of a complex series of interacting feedback loops whereby proteins regulate their own expression across day and night. Early bird (ebi) is a circadian mutation that causes the clock to speed up: ebi plants have short circadian periods, early phase of clock gene expression, and are early flowering.

The gene responsible for the ebi-1 phenotype, AtNFXL-2, is a zinc finger transcription factor, a homolog of the human NF-X1 protein. In humans, NF-X1 binds to the X-box found in class II MHC genes. Arabidopsis has two NF-X1 homologs, AtNFXL-1 and AtNFXL-2, which are thought to act antagonistically to regulate genes involved in salt, osmotic and drought stress, with AtNFXL-1 activating and AtNFXL-2 repressing stress-inducing genes. AtNFXL-1 has also been suggested to be a negative regulator of defense-related genes and temperature stress. Thus, the clock phenotype of the AtNFXL-2 mutant provides an intriguing link between the clock and biotic and abiotic stress responses. This link has been alluded to in a recent review and in the identification of a possible role for the clock protein GI in cold stress tolerance.

The circadian phenotypes of the ebi-1 mutant have been characterized by Johansson, M. et al. (2011) Partners in Time: EARLY BIRD Associates with ZEITLUPE and Regulates the Speed of the Arabidopsis Clock. Plant Physiol. 155(4): 2108-2122.

Populus trees have two EBI1 genes: EBI1a (SEQ ID NO: 1) and EBI1b (SEQ ID NO: 3) as well as two EBI2 genes: EBI2a (SEQ ID NO: 6) and EBI2b (SEQ ID NO: 8). See also Johansson et al. (2011), Supplemental Table I and Supplemental FIG. 1.

Increased plant production of biomass particularly in agriculture and forestry is of large importance for food and as a renewable resource for energy and materials fulfilling the demands of an increasing population and as a CO₂ sink for increasing levels of green house gases. At present most forest production is based on the production of boreal forests where trees are exposed to large seasonal variation in day length and temperature resulting in rather short growing seasons. To increase productivity of these and other forests it is essential to obtain germplasm that thrive at extensive latitudinal clines and produce large quantities of biomass during the most productive time.

### BRIEF DESCRIPTION OF THE DRAWINGS

FIG. 1A illustrates diurnal expression of Populus EBI1 from Real time PCR Biological repeat 1. The Y-axis is representing the relative expression (PttEBI1a/Ptt18S)

FIG. 1B illustrates the light induced diurnal expression of Populus EBI2 from Real time PCR Biological repeat 1. The Y-axis is representing the relative expression (PttEBI2a/Ptt18S).

FIG. 1C illustrates the light induced diurnal expression of Populus EBI1 from Real time PCR Biological repeat 2. The Y-axis is representing the relative expression (PttEBI1a/Ptt18S)

FIG. 1D illustrates the light induced expression of Populus EBI2 from Real time PCR Biological repeat 2. The Y-axis is representing the relative expression (PttEBI2a/Ptt18S).

FIG. 1E illustrates the diurnal expression of Populus EBI1 .The two Y-axises are representing the expression level of EBI1a and EBI1b, respectively. LDHH data were obtained from the diurnal data base [http://diurnal.cgrb.oregonstate.edu/]*

FIG. 1F illustrates the diurnal expression of Populus EBI2a. The two Y-axises are representing the expression level of EBI2a and EBI2b, respectively. The LDHH data is from the diurnal data base.

In FIG. 1A to 1F, the X-axis is representing the time in hours. Samples were taken during constant temperature and cycles of light and dark represented by white and grey bars respectively.

FIG. 1G illustrates expression of EBI1 in various Populus tissues (data from Poplar eFP Browser at http://bar.utoronto.cal).

FIG. 1H illustrates expression of EBI2 in various Populus tissues (Poplar eFP Browser).

In FIG. 1G to 1H, the tissues are mature leaf (M); young leaf (L); root (R); dark-grown seedling etiolated (S); dark-grown seedling, etiolated, exposed to light for 3 hours (S3); continuous light-grown seedling (CS); female catkins (FC); male catkins (MC) and xylem (X). The Y-axis is representing the expression level.

FIG. 2 shows elongation and radial growth in transgenic Populus trees wherein EBI1 (FIG. 2A) and EBI2 (FIG. 2B), have been knocked out down by RNA interference. T89 indicates a wild type tree. The left Y-axis is representing the height in cm. The right Y-axis is representing the diameter in mm. The X-axis is representing the time in days.

FIG. 3 illustrates the ratio (mutant/WT) of Populus EBI1 (FIG. 3A) and EBI2 (FIG. 3B) expression in transgenic trees wherein EBI1 and EBI2, respectively, have been knocked out down by RNA interference.

FIG. 4 shows seasonal growth pattern (bud set score at the Y-axis, 3=active growth, 0=dormant) in transgenic Populus trees wherein EBI1 (FIG. 4A) and EBI2 (FIG. 4B), have been knocked out down by RNA interference. The X-axis is representing the time in days under short days.

### DISCLOSURE OF THE INVENTION

It has surprisingly been found that trees with decreased levels of EARLY BIRD1 (EBI1) and EARLY BIRD2 (EBI2) transcript grow better than wild type trees. The growth phenotype is inversely proportional to the level of expressed transcript (more growth when there is less transcript) indicating that the effect is due to the down-regulation of the targeted transcripts. It is suggested that EBI genes are useful as targets for down-regulation to obtain increased growth and generating more biomass of forest trees.

Consequently, in one aspect the invention provides a method for producing a genetically modified plant with improved growth properties as compared to a corresponding non-genetically modified wild type plant, said method comprising:
(a) reducing or deleting the amount or activity of an EBI1 or EBI2 polypeptide in a plant cell, a plant or a part thereof; and
(b) generating and/or selecting a genetically modified plant with improved growth properties as compared to a corresponding non-genetically modified wild type plant and growing under conditions which permit the development of the plant.

The term “improved growth properties” should be understood as primary growth, including a lengthening of the stem and roots, as well as secondary growth of a plant, including production of secondary tissue, “wood”, from the cambium and an increase in the girth of stems and roots. One way of following the growth might be by measuring the height and the diameter of the stem and optionally calculating the volume of the stem and compare it with a wild type population or with parental control trees of the plant of interest.

In an additional aspect, the method according to the invention comprises the additional steps of:
(c) selfing or crossing the genetically modified plant with itself or another plant, respectively, to produce seed; and
(d) growing a progeny plant from the seed, wherein the progeny plant has improved growth properties.

Preferably, the said EBI1 polypeptide comprises a domain having at least about 161 amino acids, said domain being at least 75% identical, such as 80%, 85%, 90%, 95% or 100% identical, with the amino acid sequence shown as SEQ ID NO: 5. More preferably, the said EBI1 polypeptide has an amino acid sequence which is at least 75%, such as 80%, 85%, 90%, 95% or 100%, identical with the sequence shown as SEQ ID NO: 2 (EBI1a) or SEQ ID NO: 4 (EBI1b).

Preferably, the said EBI2 polypeptide comprises a domain having at least about 191 amino acids, said domain being at least 75% identical, such as 80%, 85%, 90%, 95% or 100% identical, with the amino acid sequence shown as SEQ ID NO: 10. More preferably, the said EBI2 polypeptide has an amino acid sequence which is at least 75%, such as 80%, 85%, 90%, 95% or 100%, identical with the sequence shown as SEQ ID NO: 7 (EBI2a) or SEQ ID NO: 9 (EBI2b).

In a further aspect, the invention provides a the method as describe above, comprising reducing or deleting the expression of at least one nucleic acid molecule, wherein said molecule is selected from: (a) a nucleic acid molecule encoding a EBI1 polypeptide or EBI2 polypeptide; and (b) a nucleic acid molecule having a nucleic acid sequence selected from the group consisting of SEQ ID NO: 1 (EBI1a), SEQ ID NO: 3 (EBI1b), SEQ ID NO: 6 (EBI2a); and SEQ ID NO: 8 (EBI2b).

In accordance with the present invention, the method comprise the further step of transforming regenerable cells of a plant with said nucleic acid construct or recombinant DNA construct and regenerating a transgenic plant from said transformed cell. When introducing the above DNA construct or vector into a plant cell, certain considerations must be taken into account, well known to those skilled in the art. The nucleic acid to be inserted should be assembled within a construct that contains effective regulatory elements that will drive transcription, as described above. There must be available a method of transporting the construct into the cell. Once the construct is within the cell, integration into the endogenous chromosomal material either will or will not occur.

Transformation techniques, well known to those skilled in the art, may be used to introduce the DNA constructs and vectors into plant cells to produce transgenic plants, in particular transgenic trees, with improved growth properties.

A person of skill in the art will realise that a wide variety of host cells may be employed as recipients for the DNA constructs and vectors according to the invention. Non-limiting examples of host cells include cells in embryonic tissue, callus tissue type I, II, and III, hypocotyls, meristem, root tissue, tissues for expression in phloem, leaf discs, petioles and stem internodes.

As listed above, Agrobacterium transformation is one method widely used by those skilled in the art to transform tree species, in particular hardwood species such as poplar. Production of stable, fertile transgenic plants is now a routine in the art. Other methods, such as microprojectile or particle bombardment, electroporation, microinjection, direct DNA uptake, liposome mediated DNA uptake, or the vortexing method may be used where Agrobacterium transformation is inefficient or ineffective, for example in some gymnosperm species.

Alternatively, a combination of different techniques may be employed to enhance the efficiency of the transformation process, e.g. bombardment with Agrobacterium coated microparticles or microprojectile bombardment to induce wounding followed by co-cultivation with Agrobacterium.

It will be understood, that the particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

Following transformation, transgenic plants are preferably selected using a dominant selectable marker incorporated into the transformation vector. Typically, such a marker will confer antibiotic or herbicide resistance on the transformed plants and selection of transformants can be accomplished by exposing the plants to appropriate concentrations of the antibiotic or herbicide. A novel selection marker using the D-form of amino acids and based on the fact that plants can only tolerate the L-form offers a fast, efficient and environmentally friendly selection system. An interesting feature of this selection system is that it enables both selection and counter-selection.

Subsequently, a plant may be regenerated, e.g. from single cells, callus tissue or leaf discs, as is standard in the art. Almost any plant can be entirely regenerated from cells, tissues and organs of the plant. After transformed plants are selected and grown to maturity, those plants showing altered growth properties phenotype are identified. Additionally, to confirm that the phenotype is due to changes in expression levels or activity of the polypeptide or polynucleotide disclosed herein can be determined by analyzing mRNA expression using Northern blots, RT-PCR or microarrays, or protein expression using immunoblots or Western blots or gel shift assays.

Consequently, in a further aspect the method according to the invention comprises at least one step selected from:
(a) introducing into at least one plant cell a nucleic acid molecule encoding a ribonucleic acid sequence, which is able to form a double-stranded ribonucleic acid molecule, whereby a fragment of at least 17 nucleotides (such as 18, 19, 20 or 21 nucleotides) of said double-stranded ribonucleic acid molecule has a nucleic acid sequence having at least 50% (such as 60%, 70%, 80%, 90%, or 95%) nucleic acid sequence identity to an EBI (i.e. EBI1 a, EBI1 b, EBI2a, or EBI2b) nucleic acid molecule;
(b) introducing into at least one plant cell an RNAi or antisense nucleic acid molecule, whereby the RNAi or antisense nucleic acid molecule comprises a fragment of at least 17 nucleotides (such as 18, 19, 20 or 21 nucleotides) with a nucleic acid sequence having at least 50% (such as 60%, 70%, 80%, 90%, or 95%) nucleic acid sequence identity to an EBI nucleic acid molecule;
(c) introducing into at least one plant cell a nucleic acid construct able to recombine with and silence, inactivate, or reduce the activity of an endogenous gene comprising an EBI nucleic acid molecule; and
(d) introducing or detecting a non-silent mutation in an endogenous gene comprising an EBI nucleic acid molecule.

In another aspect the invention provides a method wherein reducing or deleting of the amount or activity of an EBI1 polypeptide or EBI2 polypeptide is caused by any one of:
(a) a natural or induced mutation in an endogenous gene of the plant cell, the plant or a part thereof;
(b) T-DNA inactivation of an endogenous gene;
(c) site-directed mutagenesis or directed breeding of an endogenous gene,
   wherein said endogenous gene comprises an EBI nucleic acid molecule.

In a preferred aspect, the method according to the invention comprises:
(a) providing a vector comprising: (i) said nucleic acid molecule for introducing into at least one plant cell; (ii) a flanking nucleic acid molecule comprising one or more regulatory elements fused to said nucleic acid molecule, wherein the regulatory elements control expression of said nucleic acid molecule; and
(b) transforming at least one cell of said plant with the vector to generate a transformed plant with improved growth properties as compared to a corresponding non-transformed wild type plant.

In a further aspect, the invention provides a genetically modified, especially a transgenic, plant produced by the methods as described above. In accordance with the present invention, the transgenic plant may be a perennial plant which preferable is a woody plant or a woody species. In a useful embodiment, the woody plant is a hardwood plant which may be selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood plants from the Salicaceae family, such as willow, poplar and aspen including variants thereof, are of particular interest, as these two groups include fast-growing species of tree or woody shrub which are grown specifically to provide timber and bio-fuel.

In further embodiments, the woody plant is a conifer which may be selected from the group consisting of cypress, Douglas fir, fir, sequoia, hemlock, cedar, juniper, larch, pine, redwood, spruce and yew. In useful embodiments, the woody plant is a fruit bearing plant which may be selected from the group consisting of apple, plum, pear, banana, orange, kiwi, lemon, cherry, grapevine and fig. Other woody plants which may be useful in the present method may also be selected from the group consisting of cotton, bamboo and rubber plants. Other plants, which may be useful is grasses grown for biomass production, for example Miscanthus and Switchgrass.

The present invention extends to any plant cell of the above transgenic plants obtained by the methods described herein, and to all plant parts, including harvestable parts of a plant, seeds and propagules thereof, and plant explant or plant tissue. The present invention also encompasses a plant, a part thereof, a plant cell or a plant progeny comprising a DNA construct according to the invention. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention.

Consequently, the invention provides a genetically modified plant having improved growth properties as compared to a corresponding non-genetically modified wild type plant, wherein said plant has a reduced amount or activity of a EBI1 or EBI2 polypeptide, and wherein the genome of said plant comprises a genetic modification selected from any one of:
i) a non-silent mutation in an endogenous gene comprising a nucleic acid molecule encoding an EBI1 or EBI2 polypeptide;
ii) a transgene inserted into said genome, said transgene comprising a nucleic acid molecule encoding a ribonucleic acid sequence, which is able to form a double-stranded ribonucleic acid molecule, whereby a fragment of at least 17 nucleotides of said double-stranded ribonucleic acid molecule has a homology of at least 50% to a nucleic acid molecule encoding an EBI1 or EBI2 polypeptide;
iii) a mutation in an endogenous gene comprising a nucleic acid molecule encoding an EBI1 or

EBI2 polypeptide, induced by introducing into at least one plant cell a nucleic acid construct able to recombine with and silence, inactivate, or reduce the activity of the endogenous gene,

wherein said EBI1 polypeptide has an amino acid sequence having at least 80% amino acid sequence identity to a sequence selected from among SEQ ID NOS: 2, 4 and 5, or wherein said EBI2 polypeptide has an amino acid sequence having at least 80% amino acid sequence identity to a sequence selected from among SEQ ID NOS: 7, 9 and 10.

In another embodiment, the invention provides the use of EBI1 and EBI2 genes for the identification of plants having increased growth as compared to the wild-type.

In a further embodiment, the invention provides the use of EBI1 and EBI2 genes and polypeptides in the identification of agents useful for inhibiting EBI1 or EBI2 activity, thereby being useful for improving plant growth.

In yet a further embodiment, the invention provides the use of EBI1 and EBI2 genes as candidate genes in marker assisted breeding.

### EXAMPLES

### Example 1

### Expression Patterns of EBI1 and EBI2

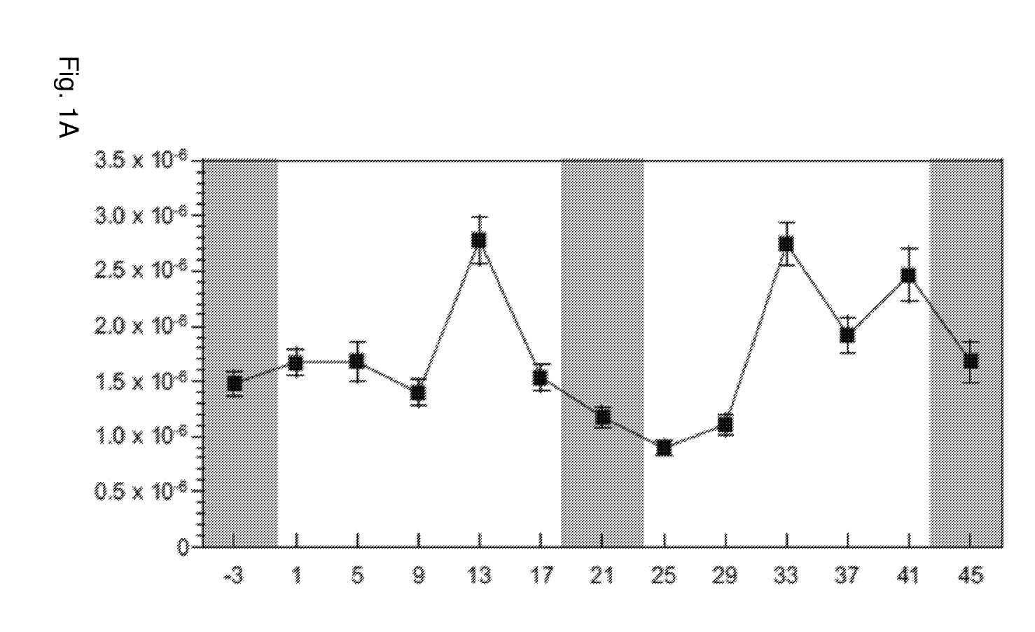

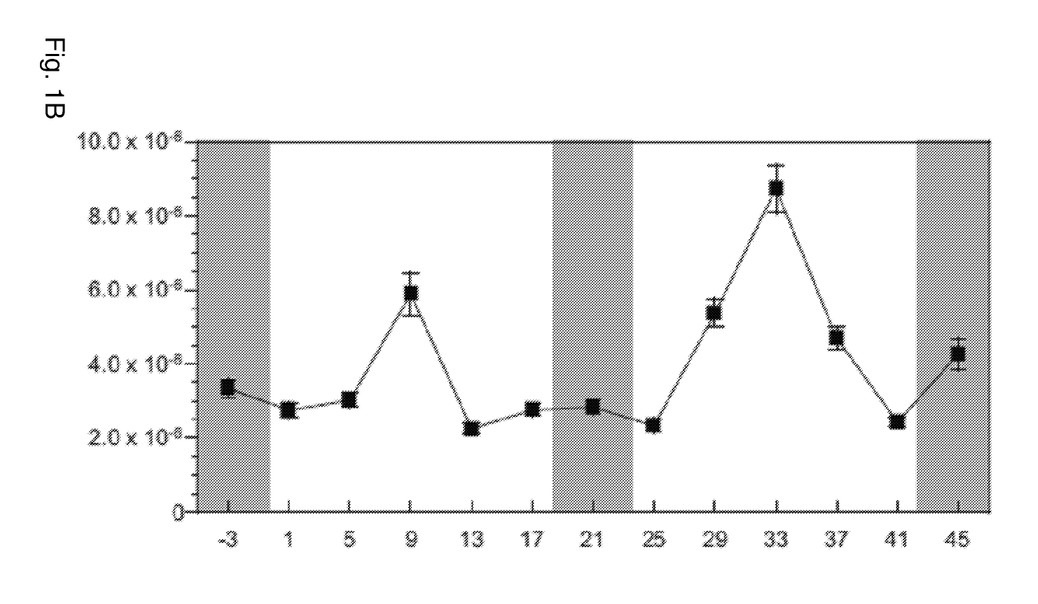

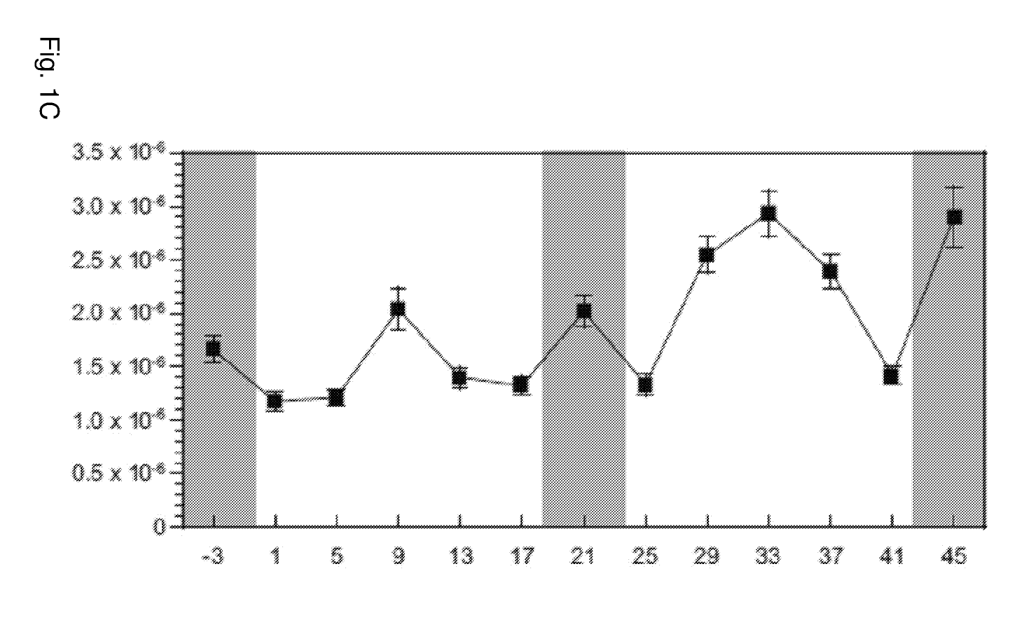

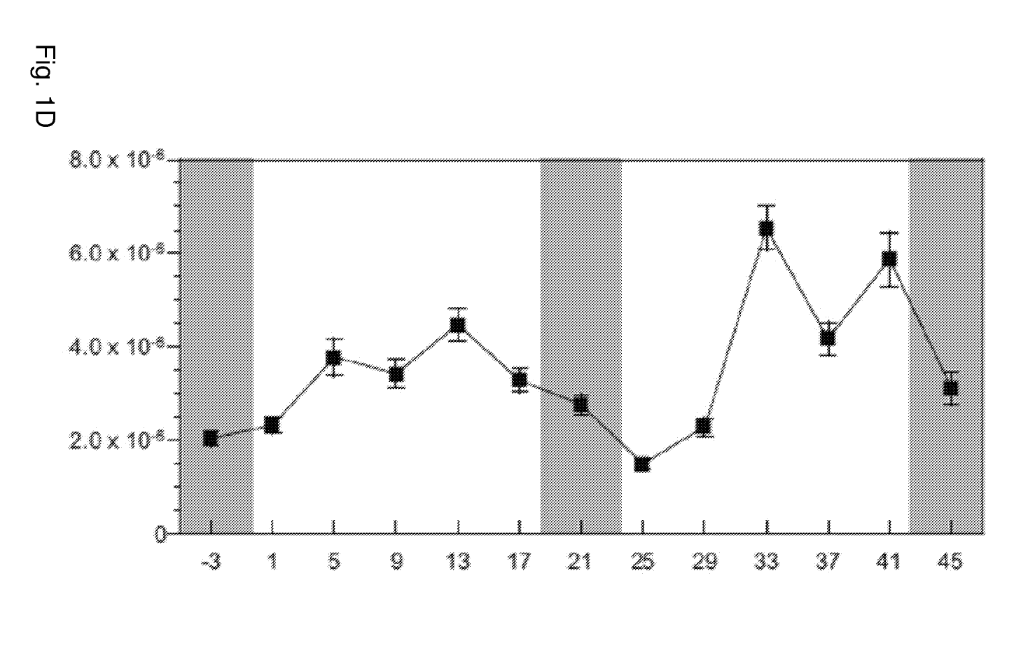

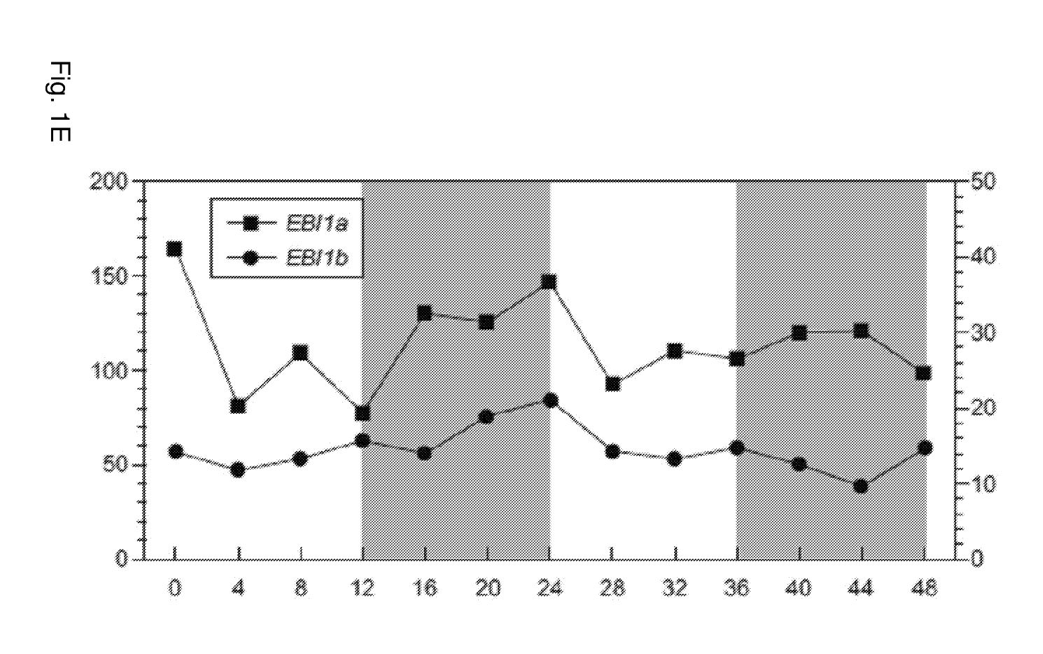

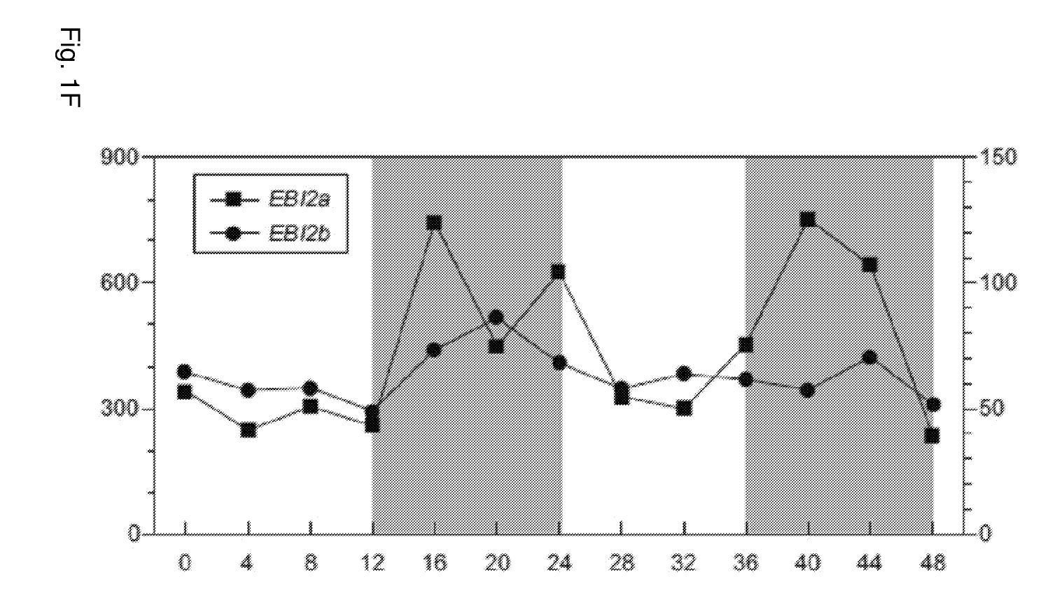

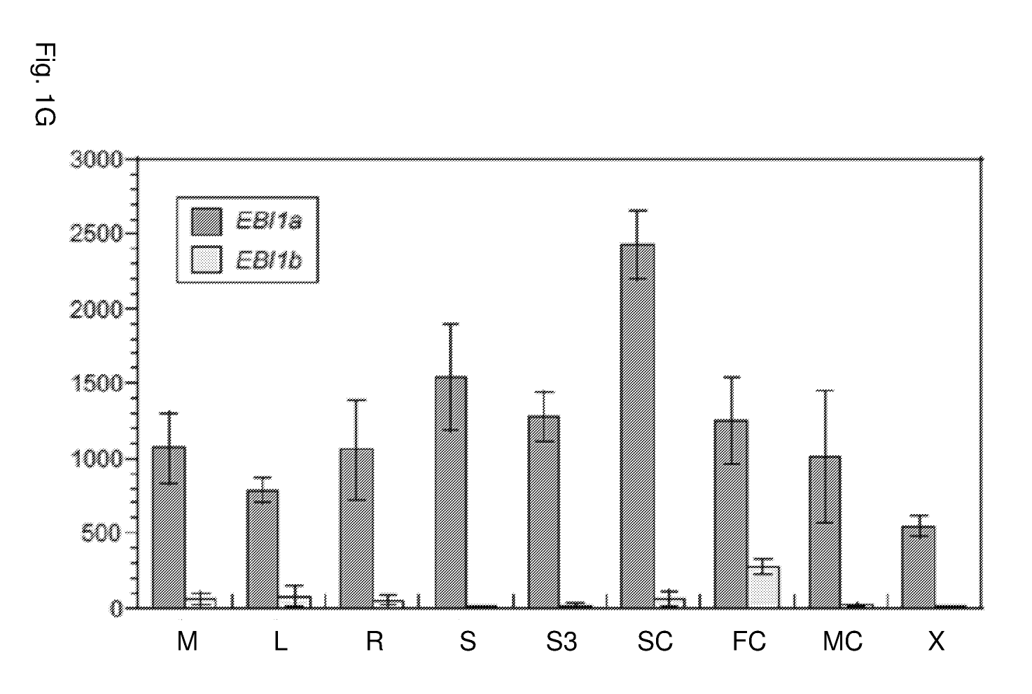

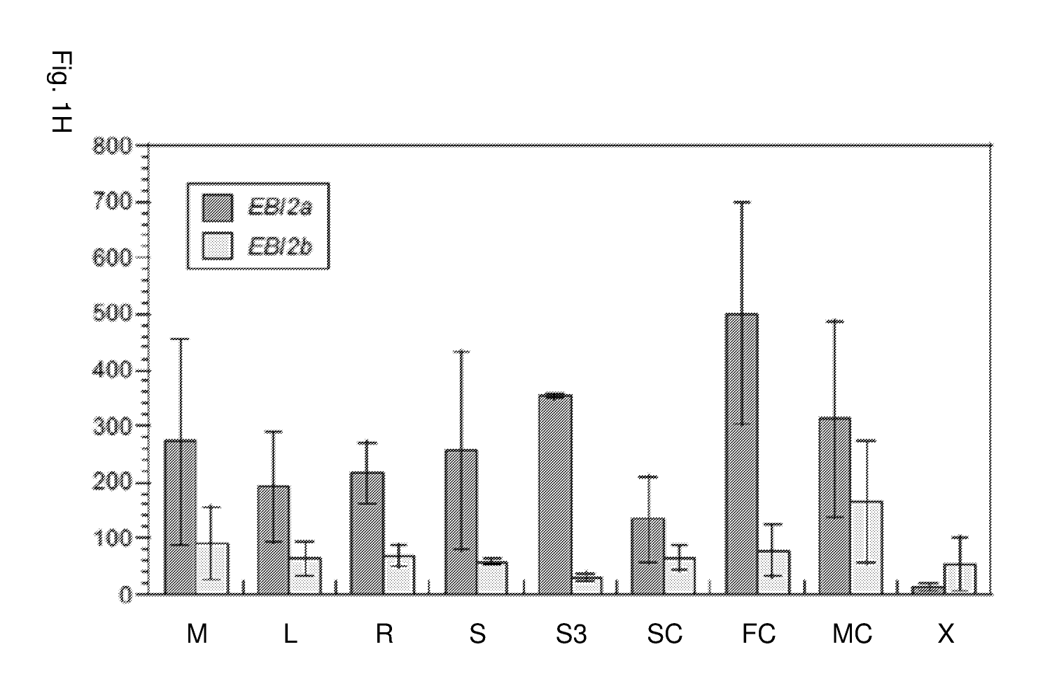

EBI1 and EBI2 appear to have a light induced and diurnal expression with a circadian pattern e.g. EBI2, less clear so EBI1, when assayed every four hours under 48 h in an 18 h light/6 h dark day length regime (18° C./18° C.) starting 3 h before dawn (FIG. 1, rows 1 and 2; Real Time PCR Biological repeat 1 and 2, each containing leaves sampled at 7-9 internodes from four independent trees at each time point) and in DIURNAL (FIG. 1, row 3; http://diurnal.cgrb.oregonstate.edu/).

As shown in FIG. 1, row 4, EBI1and EBI2 are expressed in various tissues as found in Poplar eFP Browser (http://bar.utoronto.ca/efppop/cgi-bin/efpWeb.cgi).

### Example 2

### Preparation and Growth of Transgenic Plants

RNAi trigger regions were amplified from Populus tremula×tremuloides cDNA by PCR using Platinum pfx DNA polymerase (Invitrogen, Carlsbad, Calif., USA) according to the product manual with following primer sets:

| PttEBI2 | |
|---|---|
| (SEQ ID NO: 11) | |
| Forward: | 5′-CACCGCGGCCGCCCATCTCGTGTGATTGGC-3′; |
| (SEQ ID NO: 12) | |
| Reverse: | 5′-CTTCCACGAAGTTCCCTTCAGAG-3′; |
| PttEBI1 | |
| (SEQ ID NO: 13) | |
| Forward: | 5′-CACCGCGGCCGCGGACTTGGACTTCTTCCT-3′; |
| (SEQ ID NO: 14) | |
| Reverse: | 5′-GATTCGTGGATGTCTTCTTCTGTG-3′. |

The EBI1constructs were used to down-regulate EPI1a and EPI1b, and the EBI2 constructs were used down-regulate EBI2a and EBI2b.

The PCR products were cloned in pENTR™/SD/D-TOPO® vector (Invitrogen, Carlsbad, Calif., USA). In order to remove the ineffective trigger region descended from pENTR™/SD/D-TOPO® vector, these vectors were digested with Notl and self-ligated. These entry vectors were subjected to dideoxy-nucleotide sequencing and used in the LR-Gateway reaction (Invitrogen, Carlsbad, Calif., USA) with the destination vector, pANDA35HK. Agrobacterium mediated transformation was subsequently used in order to transform hybrid aspen, Populus tremula L. ×P. tremuloides Mich. Clone T89 was transformed and regenerated according to methods known in the art.

The transgenic poplar lines were grown together with their wildtype control (wt) trees, in a growth chamber under a photoperiod of 18 h and a temperature of 18° C./18° C. (day/night). The plants were fertilized weekly Weibulls Rika S NPK 7-1-5 diluted 1 to 100 (final concentrations NO₃, 55 g/1; NH₄, 29 g/1; P, 12 g/1; K, 56 g/1; Mg 7,2 g/1; S, 7,2 g/1; B, 0,18 g/1; Cu, 0,02 g/1; Fe, 0,84 g/1; Mn, 0,42 g/1; Mo, 0,03 g/1; Zn, 0,13 g/L). Height and diameter was measured and used for analysis of growth.

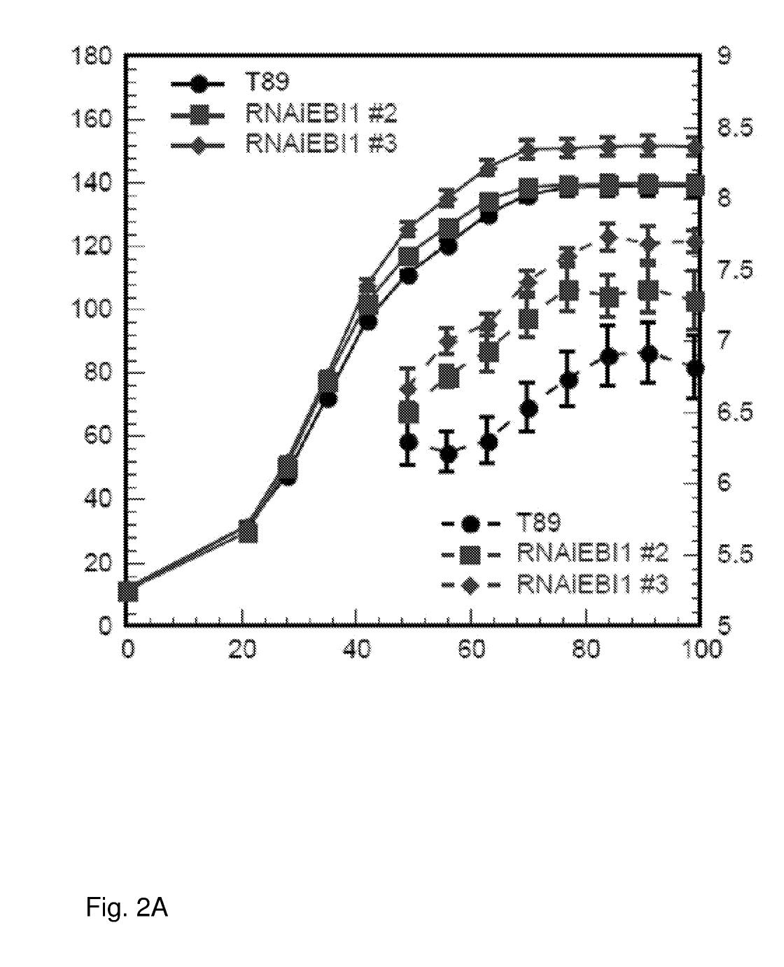

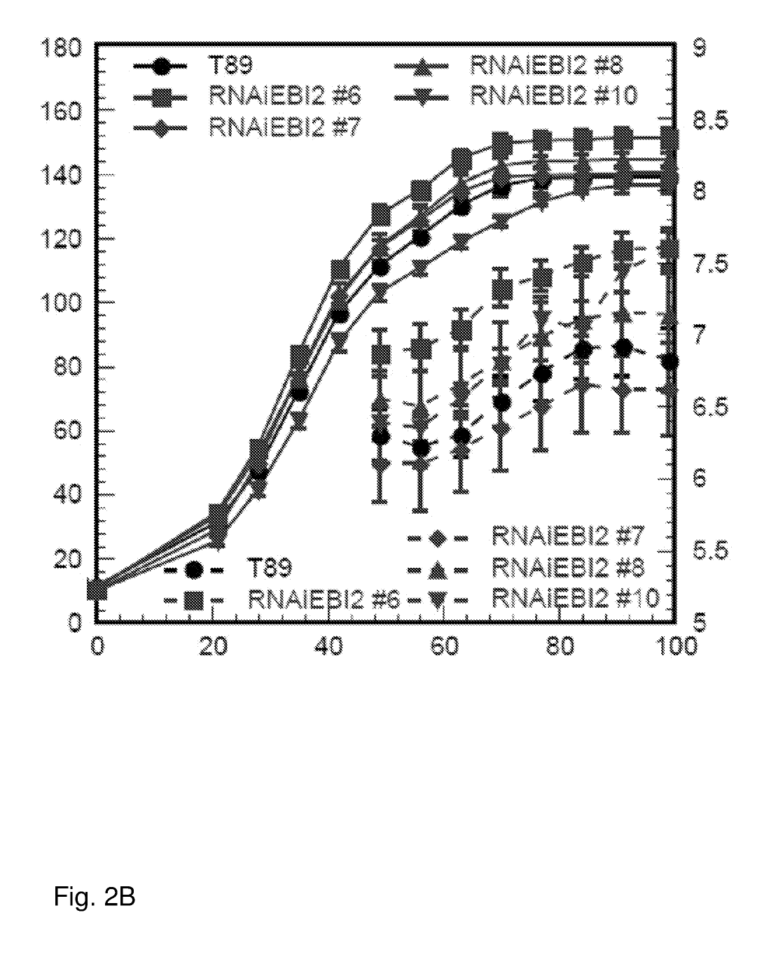

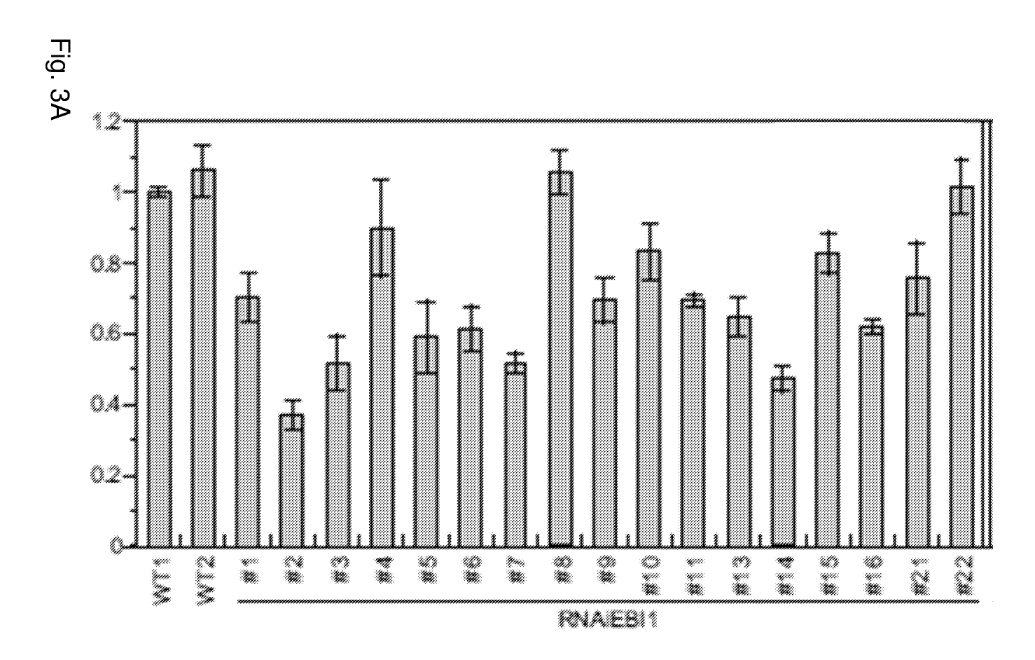

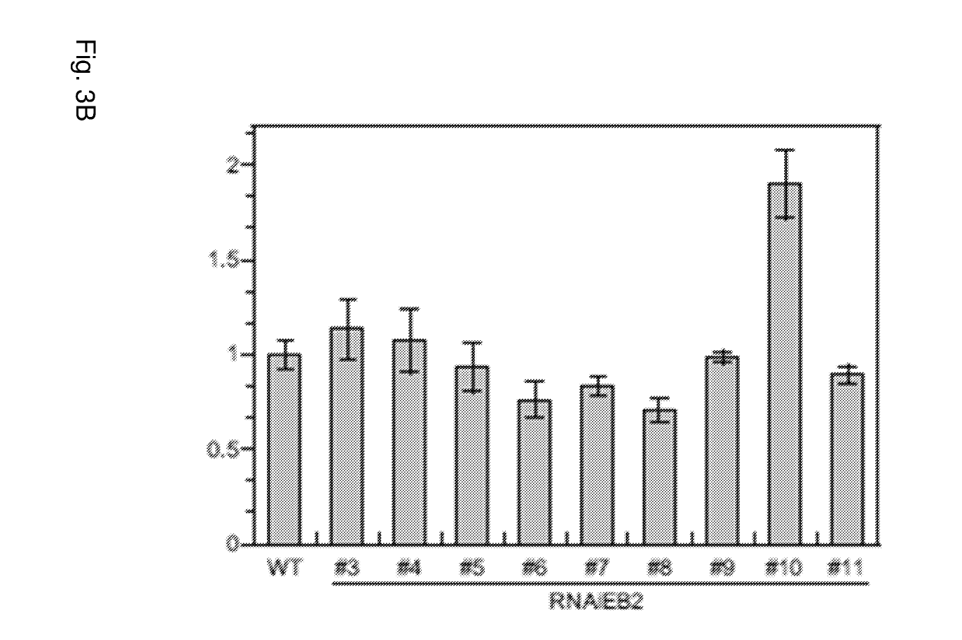

Knock-out of EBI1 and EBI2 resulted in transgenic trees with an increase in both elongation as well as radial growth compared to the wildtype T89 (FIG. 2 and Table I).

As shown in Table I, some of the EBI1 transgenic trees show 25-28% increased volume growth index and 5-13% increased height. Some of the EBI2 transgenic trees show 15-36% volume growth index and 6-14% increased height.

TABLE I
Ratios are the average of transgenic line replicates divided with
average of wt values.
Volume index are calculated as (diameter × diameter × height).
The t-test values show the p-value.

| | Ratio | t-test | | | ratio | |
|---|---|---|---|---|---|---|
| | vol | volume | ratio | t-test | di- | t-test |
| Genotype | index | index | height | height | ameter | diameter |
| RNAiEBI1 #1 | 1.12 | 0.14875 | 1.02 | 0.25863 | 1.05 | 0.1909 |
| RNAiEBI1 #2 | 1.12 | 0.099 | 1.05 | 0.0016 | 1.03 | 0.31235 |
| RNAiEBI1 #3 | 1.25 | 0.01158 | 1.13 | 0.00015 | 1.05 | 0.17114 |
| RNAiEBI1 #4 | 1.28 | 0.01273 | 1.11 | 0.00427 | 1.07 | 0.06444 |
| RNAiEBI2 #10 | 0.95 | 0.50156 | 0.93 | 0.0123 | 1.01 | 0.70251 |
| RNAiEBI2 #11 | 1.01 | 0.95361 | 1.00 | 0.86388 | 1.00 | 1.0000 |
| RNAiEBI2 #5 | 0.98 | 0.81686 | 1.02 | 0.51223 | 0.98 | 0.62944 |
| RNAiEBI2 #6 | 1.36 | 0.00414 | 1.14 | 0.00024 | 1.09 | 0.03644 |
| RNAiEBI2 #7 | 1.00 | 0.99839 | 1.05 | 0.20109 | 0.97 | 0.5426 |
| RNAiEBI2 #8 | 1.15 | 0.17487 | 1.06 | 0.01953 | 1.04 | 0.36861 |

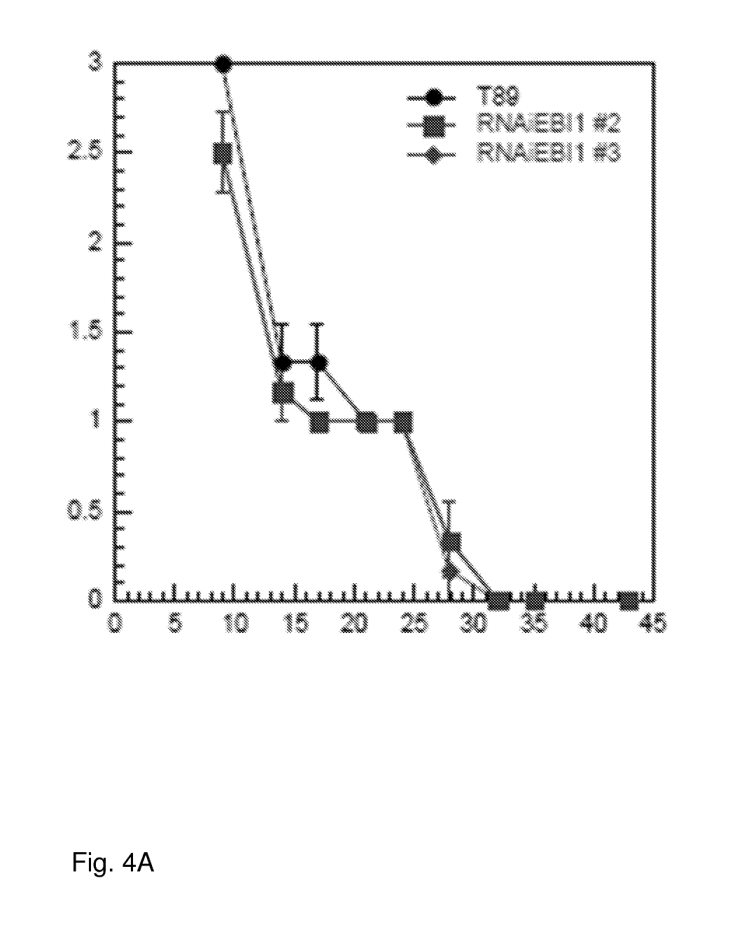

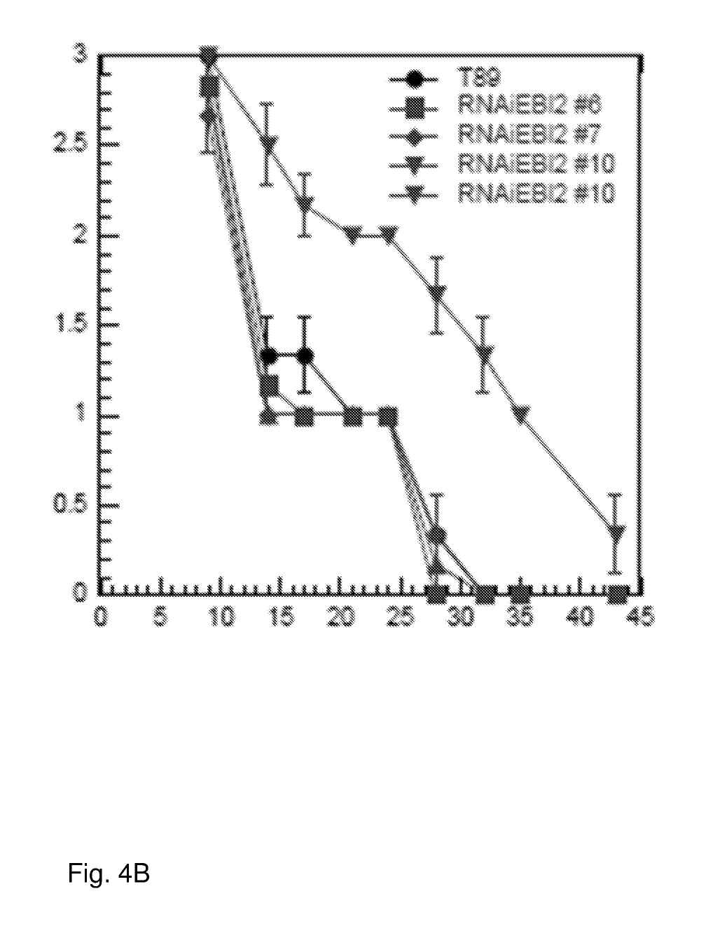

The level of gene expression was in good agreement with the observed phenotype (FIG. 3). No negative effects with respect to seasonal growth pattern, e.g. bud set, were observed (FIG. 4).

1. A method for producing a genetically modified plant with improved growth properties as compared to a corresponding non-genetically modified wild type plant, said method comprising:
a) reducing or deleting the amount or activity of an EBI1 or EBI2 polypeptide in a plant cell, a plant or a part thereof to produce a modified plant cell, plant or a part thereof;
b) generating, from the modified plant cell, plant or part thereof in step a) a genetically modified plant;
c) identifying a genetically modified plant from step b) with improved growth properties as compared to a corresponding non-genetically modified wild type plant; and
d) growing said genetically modified plant under conditions which permit the development of the genetically modified plant.

2. The method of claim 1, the method steps further comprising:
e) selfing or crossing the genetically modified plant with itself or another plant, respectively, to produce seed; and
d) growing a progeny plant from the seed, wherein the progeny plant has the improved growth properties.

3. The method of claim 1, wherein said polypeptide is an EBI1 polypeptide comprising a domain having at least about 161 amino acids, said domain being at least 80% identical with the amino acid sequence shown as SEQ ID NO: 5.

4. The method of claim 3, wherein said EBI1 polypeptide has an amino acid sequence having at least 80% amino acid sequence identity to a sequence selected from SEQ ID NOS: 2 and 4.

5. The method of claim 4, wherein said EBI1 polypeptide has an amino acid sequence selected from SEQ ID NOS: 2 and 4.

6. The method of claim 1, wherein said polypeptide is an EBI2 polypeptide comprising a domain having at least about 191 amino acids, said domain being at least 80% identical with the amino acid sequence shown as SEQ ID NO: 10.

7. The method of claim 6, wherein said subunit is a EBI2 polypeptide and wherein the amino acid sequence of the polypeptide has at least 80% amino acid sequence identity to a sequence selected from SEQ ID NOS: 7 and 9.

8. The method of claim 7, wherein said EBI2 polypeptide has an amino acid sequence selected from SEQ ID NOS: 7 and 9.

9. The method according to claim 1, comprising reducing or deleting the expression of at least one nucleic acid molecule, wherein said molecule is selected from:
a nucleic acid molecule encoding a EBI1 polypeptide or EBI2 polypeptide; and
a nucleic acid molecule having a nucleic acid sequence selected from the group consisting of SEQ ID NOS: 1, 3, 6 and 8.

10. The method according to claim 9, whereby the method comprises at least one step selected from the group consisting of:
introducing into at least one plant cell a nucleic acid molecule encoding a ribonucleic acid sequence, which is able to form a double-stranded ribonucleic acid molecule, whereby a fragment of at least 17 nucleotides of said double-stranded ribonucleic acid molecule has a nucleic acid sequence having at least 50% nucleic acid sequence identity to a nucleic acid molecule as described in claim 9;
introducing into at least one plant cell an RNAi or antisense nucleic acid molecule, whereby the RNAi or antisense nucleic acid molecule comprises a fragment of at least 17 nucleotides with a nucleic acid sequence having at least 50% nucleic acid sequence identity to a nucleic acid molecule as described in claim 9;
introducing into at least one plant cell a nucleic acid construct able to recombine with and silence, inactivate, or reduce the activity of an endogenous gene comprising a nucleic acid molecule as described in claim 9; and
introducing or detecting a non-silent mutation in an endogenous gene comprising a nucleic acid molecule as described in claim 9.

11. The method according to claim 9, wherein reducing or deleting of the amount or activity of an EBI1 polypeptide or EBI2 polypeptide is caused by any one of:
a natural or induced mutation in an endogenous gene of the plant cell, the plant or a part thereof;
T-DNA inactivation of an endogenous gene;
site-directed mutagenesis or directed breeding of an endogenous gene, wherein said endogenous gene comprises a nucleic acid molecule as described in claim 9.

12. A method according to claim 9, wherein reducing or deleting the amount or activity of an EBI1 or EBI2 polypeptide in a plant cell, a plant or a plant part thereof comprises:
providing a vector comprising: (i) said nucleic acid molecule for introducing into at least one plant cell; (ii) a flanking nucleic acid molecule comprising one or more regulatory elements fused to said nucleic acid molecule, wherein the regulatory elements control expression of said nucleic acid molecule; and
transforming at least one cell of said plant with the vector to generate a transformed plant with improved growth properties as compared to a corresponding non-transformed wild type plant.

13. The method according to claim 1, wherein the plant is a perennial woody plant.

14. The method according to claim 13, wherein the plant is a hardwood plant selected from the group consisting of acacia, eucalyptus, hornbeam, beech, mahogany, walnut, oak, ash, willow, hickory, birch, chestnut, poplar, alder, aspen, maple, sycamore, ginkgo, a palm tree and sweet gum.

15. A genetically modified plant produced by the method according to claim 1.

16. A genetically modified plant having improved growth properties as compared to a corresponding non-genetically modified wild type plant, wherein said plant has a reduced amount or activity of a EBI1 or EBI2 polypeptide, and wherein the genome of said plant comprises a genetic modification selected from any one of:
i) a non-silent mutation in an endogenous gene comprising a nucleic acid molecule encoding an EBI1 or EBI2 polypeptide;
ii) a transgene inserted into said genome, said transgene comprising a nucleic acid molecule encoding a ribonucleic acid sequence, which is able to form a double-stranded ribonucleic acid molecule, whereby a fragment of at least 17 nucleotides of said double-stranded ribonucleic acid molecule has a homology of at least 50% to a nucleic acid molecule encoding an EBI1 or EBI2 polypeptide;
iii) a mutation in an endogenous gene comprising a nucleic acid molecule encoding an EBI1 or EBI2 polypeptide, induced by introducing into at least one plant cell a nucleic acid construct able to recombine with and silence, inactivate, or reduce the activity of the endogenous gene,
wherein said EBI1 polypeptide has an amino acid sequence having at least 80% amino acid sequence identity to a sequence selected from among SEQ ID NOS: 2, 4 and 5, or wherein said EBI2 polypeptide has an amino acid sequence having at least 80% amino acid sequence identity to a sequence selected from among SEQ ID NOS: 7, 9 and 10; and
wherein said genetically modified plant has improved growth properties as compared to a corresponding non-genetically modified wild type plant.
